(19) United States
(12) Patent Application Publication
(10) Pub. No.: US 2009/0305423 A1
(43) Pub. Date: Dec. 10, 2009

(54) Methods for Monitoring Composition and Flavor Quality of Cheese Using a Rapid Spectroscopic Method

(75) Inventors: Anand S. Subramanian, Columbus, OH (US); Luis E. Rodriguez-Saona, Dublin, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: Ohio State University Research Foundation, Columbus, OH (US)

(21) Appl. No.: 12/481,278

(22) Filed: Jun. 9, 2009

Related U.S. Application Data
(60) Provisional application No. 61/150,348, filed on Feb. 6, 2009.
(60) Provisional application No. 61/059,890, filed on Jun. 9, 2008.

(51) Int. Cl. G01N 33/04

(52) U.S. Cl. 436/22

(57) ABSTRACT

Methods for evaluating cheese comprising using a rapid extraction method and an IR spectra analysis of the cheese are disclosed.

### CROSS-REFERENCE TO RELATED APPLICATIONS

This application claims the benefit of U.S. Provisional Application Nos. 61/059,890, filed Jun. 9, 2008, and 61/150,348 filed Feb. 6, 2009, the disclosures of which are expressly incorporated herein by reference.

### STATEMENT REGARDING FEDERALLY SPONSORED RESEARCH

This invention was made with no Government support and the Government has no rights in this invention.

### TECHNICAL FIELD AND INDUSTRIAL APPLICABILITY OF THE INVENTION

This invention is directed to methods for evaluating cheese comprising using a rapid extraction process and an IR spectra analysis of the cheese.

### BACKGROUND OF THE INVENTION

About 9.13 billion pounds of cheese are produced in the US every year, of which 34% is Cheddar cheese (NASS, 2007). Cheese composition and flavor quality, which influence the consumer acceptance, price and food processing application, develop during the ripening process. Cheese ripening or maturation is a slow process (2-24 months) characterized by a series of complex physical, chemical, and microbiological changes affecting the principal components of the cheese (Singh et al., 2003). The complex nature of the cheese ripening process and heterogeneous nature of cheese make it a challenge to produce cheese of uniform composition and sensory properties, especially flavor.

Cheese flavor is due to a balance of concentration and ratios of various classes of compounds (Kosikowski and Mucquot, 1958; McSweeney and Sousa, 2000).

The effect of the manufacturing process, the composition of milk (such as protein and fat level), and the biochemical events that occur during ripening will influence the composition and final quality of the cheese (Chen et al., 1998). Furthermore, the composition and flavor profile of cheese are complex and variety- or type-specific (Akalin et al., 2002) and are determined to a great extent by breakdown of proteins, fats and carbohydrates during ripening (Singh et al., 2003; Bachmann et al., 1999). The principle compounds that contribute to the flavor include organic acids, amino acids, sulfur compounds, lactones, methyl ketones, alcohols and phenolic substances (Seitz, 1990; Urbach, 1993). Currently, cheese components such as fat, moisture, pH and salt content are determined using standard chemical and chromatographic methods while the flavor by trained taste panels. These approaches are complex, labor-intensive, expensive and time-consuming, thereby hindering quality control efforts.

Attempts have been directed at finding and evaluating microbiological and biochemical parameters by which cheese could be classified and whereby uniform cheese quality could be established (Singh et al., 2003; Adda et al., 1982; Farkye and Fox, 1990; Seitz, 1990). Methods such as high performance liquid chromatography (Lues and Bekker, 2002; Akalin et al., 2002), gas chromatography (Partidario et al., 1998; Thierry et al., 1999) and mass spectrometry (Alli et al., 1998) have been investigated for analysis of cheese components. These instrumental methods have made significant contributions to the understanding of the ripening process. However, they are complicated, time-consuming, require different conditions and accessories for analyzing different classes of compounds, and have limited applications as routine quality control methods. Thus, for at least these reasons, no reliable instrumental methods exist for rapid analysis of flavor quality. Furthermore, it is practically difficult to taste and evaluate each batch of cheese.

The “component balance theory” put forth by Kosikowski and Mucquot (1958), suggests that Cheddar cheese flavor is produced by a correct balance and concentration of a wide range of sapid and aromatic compounds. Hundreds of compounds have been identified in cheese flavor (McSweeney and Sousa, 2000).

Hence, an instrumental method that is capable of simultaneously monitoring multiple compounds and their functional groups is necessary. Furthermore, cheese composition is very complex with many interfering components. Sample heterogeneity is also very common.

Due to the above reasons, no reliable instrumental methods exist for rapid analysis of flavor quality.

Considering the above-mentioned, there is a need for rapid and reliable instrumental methods for simultaneous determination of composition and flavor quality of cheese.

There is a further need for a rapid method apart from saving time and money for the cheese industry that will also help in ensuring better product quality and safety and understanding cheese ripening.

There is a further need for an instrumental method that is capable of simultaneous monitoring of multiple compounds and their functional groups in order to determine flavor quality.

Hence, there is a need for rapid and reliable instrumental methods to determine the flavor quality of cheese. A rapid method apart from saving time and money for the cheese industry will also help in ensuring better product quality.

### SUMMARY OF THE INVENTION

In a first aspect, there is provided herein a method for evaluating a sample cheese comprising determining one or more IR spectra of the cheese at least one point in time.

In another aspect, there is provided herein a method for evaluating a sample cheese comprising using a rapid solvent extraction method in combination with Fourier transform infrared spectroscopy.

In certain embodiments, the cheese is classified based on flavor quality.

In certain embodiments, the extraction method provides water soluble extracts that have one or more well-defined and consistent spectra.

In certain embodiments, the FR-IR spectroscopy is used to identify and analyze minor components of the sample that contribute to the sample's flavor.

In certain embodiments, the extraction includes preparing the sample using water, chloroform, and ethanol.

In certain embodiments, method includes: i) extracting of water-soluble components from the sample; ii) precipitating complex proteins from the water soluble components to obtain a supernatant portion; and iii) conducting an FT-IR analysis on the supernatant portion.

In certain embodiments, extraction with organic solvents enables removal of compounds that interfere with detection of compounds that contribute to the flavor.

In certain embodiments, the detected compounds include one or more of organic acids and amino acids.

In certain embodiments, the method includes mixing ground pieces of the sample with water; optionally sonicating the mixture to break down clumps of the sample; adding chloroform to the cheeses sample-water mixture sufficient to separate complex fat in the sample from remaining portions of the sample; mixing and optionally centrifuging; mixing a resultant supernatant with ethanol sufficient to precipitate complex proteins from the supernatant; centrifuging a resultant test sample; and using the test sample in a FT-IR analysis.

In certain embodiments, the FT-IR analysis detects one or more of: asymmetric and symmetric stretching vibrations of C—H groups in methylene groups of long-chain fatty acids in the region 3,000 to 2,800 cm-1; signals from C═O groups of fatty acid esters at 1,740 cm⁻¹; amide I and amide II bands of proteins at around 1,640 and 1,540 cm-1; bands in the region 1,800 to 900 cm-1, due to C—H bending, C—O—H in-plane bending, and/or C—O stretching vibrations of lipids, organic acids, amino acids, and carbohydrate derivatives.

In certain embodiments, the FR-IR spectra is used to correlate specific flavor notes such as fermented, sour, and/or unclean, and differentiated using multivariate classification models.

In certain embodiments, the analysis time, including sample preparation time, is about 20 min or less per sample.

In certain embodiments, the method is used to detect differences in the composition of cheese from different production plants.

In certain embodiments, the specific flavor notes are correlated to the infrared spectra and the cheese is classified based on its flavor quality.

In certain embodiments, the method simultaneously determines one or more of: age, pH, fat, moisture, salt, and flavor quality of the cheese.

In certain embodiments, the method further includes preparing a sample of the cheese sample by: i) grinding the sample with liquid nitrogen; ii) extracting and centrifuging the sample, and iii) collecting a water soluble fraction.

In certain embodiments, the method further includes one or more of: drying of an extract of the sample on crystal to result in formation of a uniform film of sample; and collecting the FTIR spectra of the extract in the mid-infrared region (4000-700 cm-1), optionally using a 3-bounce zinc selenide ATR crystal.

In certain embodiments, the FTIR spectra reflect the total chemical composition of the cheese extract, with absorbance bands due to acids, esters, alcohols and peptides.

In certain embodiments, raw spectra data are transformed into second derivatives to remove baseline shifts, improve band resolution, and reduce noise and variability between replicates.

In another broad aspect, there is provided herein a method for providing quality control and control over the ripening process of cheese to achieve a cheese of desired flavor quality comprising evaluating the cheese using IR spectra of the cheese.

In another broad aspect, there is provided herein a method for classifying cheese based on its flavor quality.

In another broad aspect, there is provided herein a method rapid, cost-effective, and easy-to-use quality control method for determination of cheese composition and flavor quality comprising using the method of claim 1.

In another broad aspect, there is provided herein a method for determining of marketability of cheese early in a ripening process of the cheese, comprising evaluating the cheese using IR spectra of the cheese

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1: Raw (A and C) and derivatized (B and D) spectra of Cheddar cheese (-) and Cheddar cheese extract (- - -). Certain important functional groups and their region of absorbance are highlighted. Spectral data were obtained in the mid-infrared region (˜4000 to ˜700 cm⁻¹) at resolution of 8 cm⁻¹ by co-adding 128 scans. Cheddar cheese sample was obtained by pressing 0.5 g of cheese on a diamond ATR crystal. Extracts were scanned by drying 10 μL of the extract on a zinc selenide ATR crystal.

FIG. 2: Soft independent modeling of class analogy (SIMCA) classification plot of samples from the two production plants. The mid-infrared spectra were transformed into their 2^{nd} derivative, mean-centered and normalized prior multivariate analysis. The samples clustered distinctly indicating marked difference in the chemical composition of samples from the two production plants. The 95% probability cloud indicates the probability of a sample belonging to the cluster in which it is located.”

FIG. 3: Soft independent modeling of class analogy (SIMCA) classification plot of samples from Plant #1. The samples were projected against the first three principal components (PC) that explained the largest amount of variance among the samples. All the five samples formed distinct clusters in 3D space based on their flavor quality descriptors (1—Fermented, 2—Unclean, 3—Slight Sour, 4—Good Cheddar, and 5—Slight Burnt).

FIG. 4: Soft independent modeling of class analogy (SIMCA) classification plot of samples from Plant #2. The orientation of the clusters in the SIMCA plot correlated with the flavor of the cheese sample (Sample 6 and 14—Good Cheddar, Sample 7—Fermented, Sample 8, 9, 10, and 11—Sour and Slight Acid, Sample 12—Slight Low Flavor and Slight Sour, Sample 13—Low Flavor, and Sample 15—Sulfide.)

FIG. 5: Discriminating power plot for classification of Cheddar cheese samples. The regions of the FT-IR spectra that contribute to the discrimination of the cheese samples based on their flavor are highlighted. The higher the discriminating power at a particular wavenumber the greater is the difference between the samples in the functional group associated with that wavenumber.

FIG. 6: Typical raw (- - - -) and 2^{nd} derivative (-) FTIR spectra of Cheddar cheese extract. Exactly 7.5 μL of the extract was dried on zinc selenide crystal and scanned in the mid-infrared region (4000 to 700 cm⁻¹). Important functional groups and their region of absorbance are highlighted.

FIG. 7: Partial least squares regression (PLSR) models for prediction of FIG. 7A —pH, FIG. 7B—fat, FIG. 7C—salt, FIG. 7D—moisture, and FIG. 7E—flavor quality score of cheese samples. The spectra of 67-days old cheese were transformed into their second-derivative, mean-centered and normalized prior to multivariate analyses.

FIG. 8. Soft independent modeling of class analogy (SIMCA) classification plot for discrimination of cheese samples based on flavor quality: 1 and 2—both slight acid and good creamy, 3 and 4—both slight whey taint and medium creamy, 5 and 6—both good creamy cheddar, and 7 and 8—low flavor and good creamy. The samples were projected against the first three principal components (PC) that explained the largest amount of variance among the samples.

FIG. 9: Discriminating power plot for classification of Cheddar cheese samples. The regions of the FTIR spectra that contributed to the discrimination of the cheese samples based on their flavor are highlighted. Higher the discriminating power at a particular wavenumber the greater is the difference between the samples in the chemical groups associated with that wavenumber.

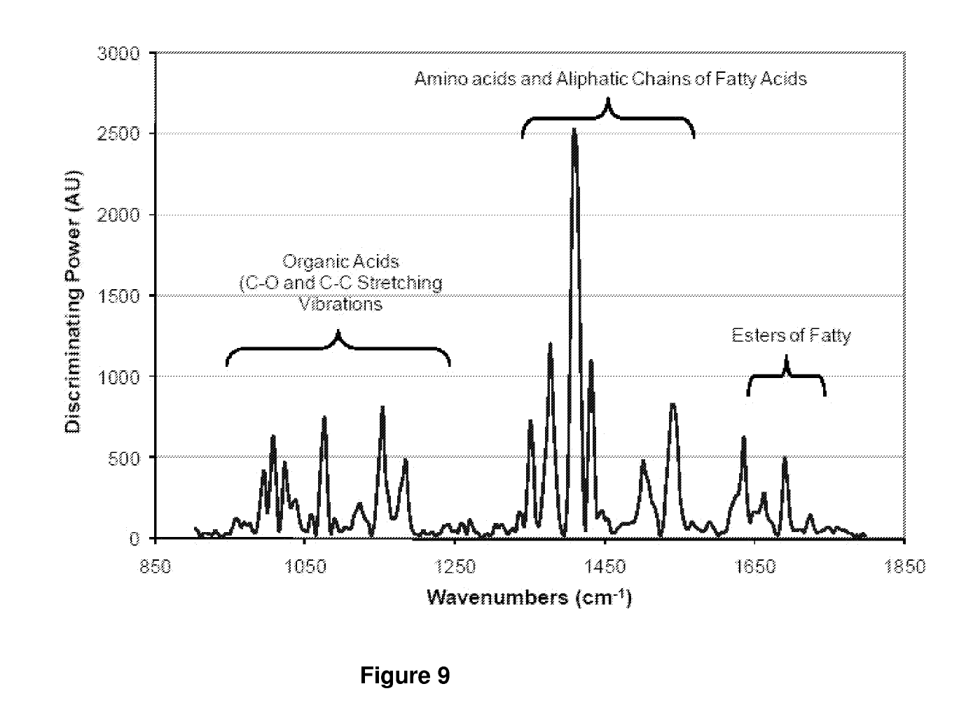

FIG. 10: Biochemical changes in Cheddar cheese that occurred between days 7 and 15 (blue), 15 and 30 (purple), 30 and 45 (red), and 45 and 73 (green) of ripening. Higher the discriminating power the greater is the change. The period from day 15 to day 30 (secondary axis) exhibited greatest change during the ripening process.

FIG. 11: Correlation between the Predicted and Actual flavor quality scores at the end of 67-days of ripening. The expected flavor quality scores were predicted using the spectra of 30-day old samples.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Fourier transform infrared (FT-IR) spectroscopy is an easy-to-use, rapid and reliable useful in the method described herein for analysis of food components. FT-IR is based on the principle that different chemical functional groups require different amounts of energy (different wavelengths) for excitation. FT-IR spectroscopy (˜4000 to ˜700 cm⁻¹) monitors the absorbance of infrared light by functional groups in the sample to provide a chemical fingerprint (spectrum) of the sample that shows the overall chemical composition of the sample. FT-IR spectroscopy combined with multivariate analysis has been suggested for rapid analysis of cheese by many researchers. Examples of its applications in cheese analysis include determination of fat, moisture and protein analysis (McQueen et al., 1995; Chen et al., 1998; Chen and Irudayaraj, 1998), shelf-life analysis of Crescenza cheese (Cattaneo et al., 2005), compositional analysis of Swiss cheese (Rodriguez-Saona et al., 2006) and determination of geographic origin of cheese (Karoui et al., 2004).

However, analysis of cheese flavor by spectroscopy and simultaneous analysis of cheese composition and flavor quality has not been explored. This is mainly because of difficulties in the sampling procedures and heterogeneous nature of cheese (McQueen et al., 1995). Furthermore, interference from other cheese constituents is a challenge that needs to be overcome for efficient FT-IR analysis.

Hence, an effective sample preparation method is essential for analysis of flavor compounds in cheese.

The present invention relates, at least in part, to a method for developing a suitable sample preparation and FT-IR spectroscopy that enables simultaneous analysis of cheese composition and flavor quality and for developing multivariate statistical models using the spectra to determine composition and flavor quality of Cheddar cheese. Described herein is a rapid and simultaneous analysis of composition and flavor quality of cheese using FT-IR spectroscopy.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

### Example I

Almost all studies on analysis of cheese by FT-IR have been directed towards monitoring major food components such as protein and fat. Limited research has been conducted on the use of FT-IR spectroscopy for analysis of minor components that contribute to the flavor. This is mainly because of difficulties in the sampling procedures and heterogeneous nature of cheese (McQueen et al., 1995). Koca et al. (2007) reported a correlation coefficient of >0.90 between acetic, propionic and butyric acid contents and the FT-IR spectra. Due to interferences from fat and protein, a complex water soluble extraction method was followed to prepare the samples for FT-IR analysis.

In another aspect, there is provided herein a sample preparation procedure that provides water soluble extracts that have well defined and consistent spectra, which when analyzed through multivariate statistical classification method, can rapidly classify cheese based on flavor quality.

Materials and Methods of Example I

Cheddar Cheese Samples

Fifteen different Cheddar cheese samples, ripened for 70 days, were obtained from two production plants of a commercial cheese manufacturer. Five cheese samples were obtained from a commercial cheese manufacturing facility (Plant #1) and 10 samples were obtained from another production plant (Plant #2). The sensory flavor quality of these cheeses was earlier analyzed by trained quality assurance personnel in the production facilities and provided along with the cheese samples. Cheese samples were vacuum-packaged and stored at −40° C. until analysis.

Extraction of Flavor Compounds for FT-IR Analysis

About 20 g of frozen cheese sample was cut into small pieces. The pieces were mixed with about 20 mL of liquid nitrogen to freeze them rapidly. The hard frozen pieces were cryogenically ground in a blender to powder them. The powered cheese was then kept frozen (−40° C.) until extraction. For extraction exactly 0.1 g of the powdered cheese was weighed and 0.5 mL of distilled water was added and mixed well. The mixture was sonicated using an ultrasonic dismembrator (Fisher Scientific, Pittsburgh, Pa.) for 10 sec to breakdown clumps and improve extraction of water soluble components from cheese powder. Equal amount (0.5 mL) of chloroform was added to the cheese powder-water mixture (to separate complex fat), mixed well and centrifuged at 15,700×g at 25° C. for 3 min. Exactly 200 μL of the resulting supernatant was mixed with equal amount of 100% ethanol (to precipitate complex proteins), and centrifuged at 15,700×g at 25° C. for 3 min. Supernatant (100 μL) was used for FT-IR analysis.

FT-IR Spectroscopy

FT-IR spectroscopy of the cheese samples was carried out on a Varian 3100 FT-IR spectrometer (Varian Inc., Palo Alto, Calif.) equipped with PERMAGLOW™ mid-IR source, extended range potassium bromate infrared beam splitter and deuterated triglycine sulfate detector. Aliquots (10 μL) of the extracts were placed on a 3-bounce MIRacle™ attenuated total reflectance (ATR) accessory with a zinc selenide crystal (Pike Technologies, Madison, Wis.) and vacuum dried to form a thin film. Infrared spectra were recorded between 4000 and 700 cm⁻¹ at a resolution of 8 cm⁻¹, using the data collection software (Varian Resolutions Pro v4.05, Varian Inc., Palo Alto, Calif.). In order to improve the signal to noise ratio 128 scans were averaged for each spectrum. For each cheese, three samples were collected from different locations and powdered. Five independent extractions were made for each powder and 3 spectra were collected per extract. Hence, for the 5 samples from Plant #1, a total of 225 (5 cheeses×3 powders per cheese×5 extractions per powder×3 spectra per extract) spectra were collected. For the 10 samples from Plant #2, a total of 450 spectra were collected.

Multivariate Analysis

Multivariate analyses of the data were carried out using a commercially available comprehensive chemometrics modeling software called Pirouette® (v3.11, Infometrix Inc., Woodville, Wash.). For analysis, spectra were imported into Pirouette®, mean-centered, transformed into their second derivative using a Savitzky-Golay polynomial filter (five-point window) and vector-length normalized. For classifying the cheese samples based on their sensory flavor quality, the spectra were analyzed by soft independent modeling of class analogy (SIMCA). SIMCA is a classification algorithm based on principal component analysis. Based on specific fingerprint spectral information from infrared light absorbance by functional groups in cheeses, multivariate analysis modeled the relationships between large numbers of dependant variables to classify different cheeses. The data were projected onto the first three principal component axes to visualize clustering of samples in 3D space based on their flavor quality (fermented, sour, good cheddar, etc.). The spectral regions influencing the classification of the cheeses were determined from the measure of variable importance (discriminating power).

Results And Discussion For Example I

FT-IR Spectra of Cheddar Cheese

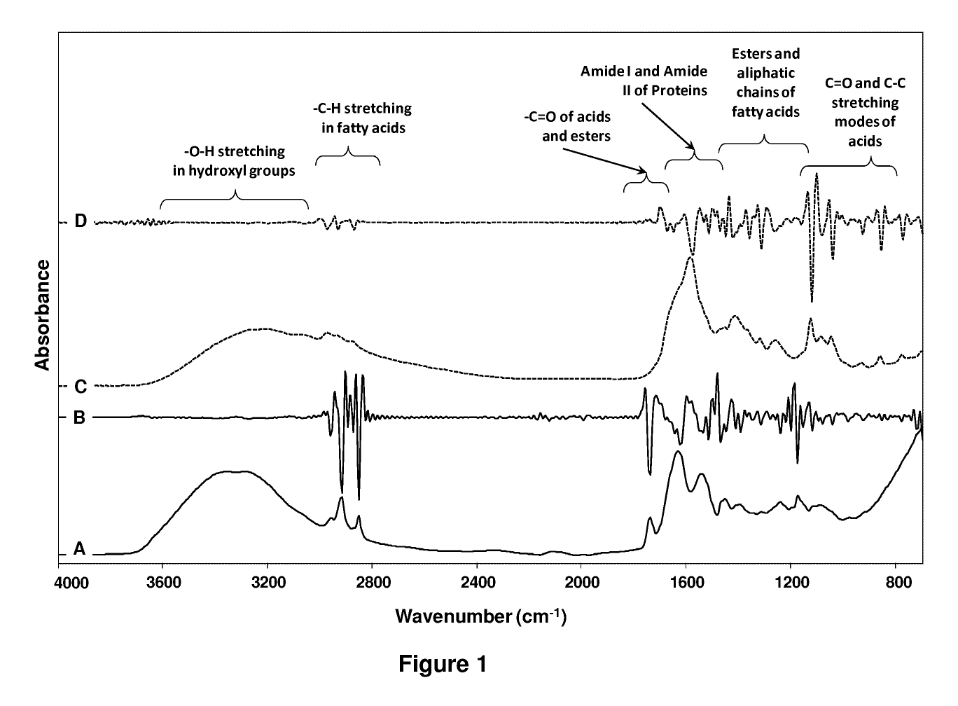

FT-IR spectra of Cheddar cheese samples were collected in the mid-infrared region (4000 to 700 cm⁻¹), using a 3-bounce ATR crystal. In a 3-bounce crystal, infrared light bounces on the sample 3 times, increasing the absorbance and hence the signal. For analysis the raw spectra were normalized, standardized and transformed to their second derivatives to remove the baseline shifts, improve the peak resolution, and reduce the variability between replicates (Kansiz et al., 1999). A typical mid-infrared spectrum of Cheddar cheese and its second derivative are shown by lines A and B, respectively in FIG. 1.

The spectra of Cheddar cheese were marked by absorbance from complex fat and protein in the regions 3,000 to 2,800 and 1,800 to 1000 cm⁻¹, respectively, which were very similar to observations reported by several other researchers (Koca et al., 2007; Rodriguez-Saona et al., 2006; Chen and Irudayaraj 1998; Chen et al., 1998). Asymmetric and symmetric stretching vibrations of C—H groups in methylene groups of long chain fatty acids were observed in the region 3000 to 2800 cm⁻¹. Strong signal from the C═O groups of fatty acid esters were also present at 1740 cm⁻¹. Broad Amide I and Amide II bands of proteins peaked at around ˜1640 and ˜1540 cm⁻¹, respectively. The spectra also included several other bands in the region 1800 to 900 cm⁻¹, primarily due to C—H bending, C—O—H in-plane bending and C—O stretching vibrations of lipids, organic acids, amino acids, and carbohydrate derivatives, that play a significant role in cheese flavor. The region from 1800 to 900 cm⁻¹ exhibited poor peak definition due to multiple absorptions and signal masking by major compounds. In order to analyze Cheddar cheese flavor, interferences and variability caused by macromolecules such as protein and fat have to be minimized and peak definition has to be improved. This emphasized the need for an extraction method to improve spectral information.

FT-IR Spectra of Water Soluble Fractions

Water soluble fractions of Cheddar cheese has been reported to contribute significantly more to Cheddar cheese flavor intensity than complex fat, fat soluble volatiles or insoluble fractions by several researchers (Engel et al., 2002; Engels et al., 1997; Aston and Creamer, 1986). Several different extraction procedures with various solvents were investigated in order to obtain consistent spectra that would enable classification of the Cheese based on flavor. Extraction using water, chloroform and ethanol was found to provide extracts of consistent composition that enabled effective discrimination of Cheddar cheese based on flavor. Drying of the extract on the crystal resulted in the formation of a uniform film of sample. The presence of ethanol in the extract helped in faster drying of the samples on the crystal. The drying time per sample was 3 min. This sample preparation method allowed for the collection of high-quality spectra with distinct spectral features that were very consistent within each sample.

FT-IR raw and derivatized spectra of the Cheddar cheese extract are shown in FIG. 1 (Lines C and D). Complex fat and protein that play a limited role in the flavor of Cheddar cheese were removed by sequential extraction with chloroform and ethanol. Chloroform extracted long chain fatty acids from the cheese-powder mixture. This was confirmed by the signal reduction in the region 3000 to 2800 cm⁻¹ and 1740 cm⁻¹ in the spectra of cheese extract (FIG. 1).

Ethanol with its high affinity to water removed water from the protein causing proteins to form complexes and precipitate on centrifugation. As shown in FIG. 1, the absorption from Amide I and Amide II bands of proteins reduced significantly in the spectra of cheese extract.

Additionally, ethanol helped in extracting compounds with slight non-polarity, including short and medium chain fatty acids, from the cheese. The resulting extract contained water soluble components from the cheese which included organic acids, short and medium chain fatty acids and their esters, alcohols, amino acids, and small peptides, all of which are known to contribute significantly to cheese flavor (Singh et al., 2003; Seitz, 1990; Urbach, 1993). This extraction procedure significantly reduced interfering compounds and improved spectral definition in the region 1800 to 900 cm⁻¹ that contains signals from several flavor-related compounds.

Classification of Cheddar Cheese Based on Flavor

Direct examination of Cheddar cheese had low discrimination and inconsistent spectra presumably due to the presence of interfering compounds and heterogeneity in cheese samples (data not shown). Visual comparison of the raw spectra of Cheddar cheese water soluble fractions showed numerous differences between cheeses in the spectral regions 1800 to 900 cm⁻¹. Using the derivatized spectra, classification models based on SIMCA were developed to discriminate cheese from the two production plants. The SIMCA models were developed by computing a small number of orthogonal variables (the principal components or PCs) that explained as much of the variation as possible between the samples, while preserving the relevant information and eliminating random noise (Mark, 2001). SIMCA classification plot, a projection of the original data onto first three principal components, allowed the visualization of well-separated clustering among the samples, whose orientation in 3D space correlated with their flavor quality. SIMCA also provides a 95% probability cloud, which means that there is 95% probability that the samples within that cloud belong to the same flavor category.

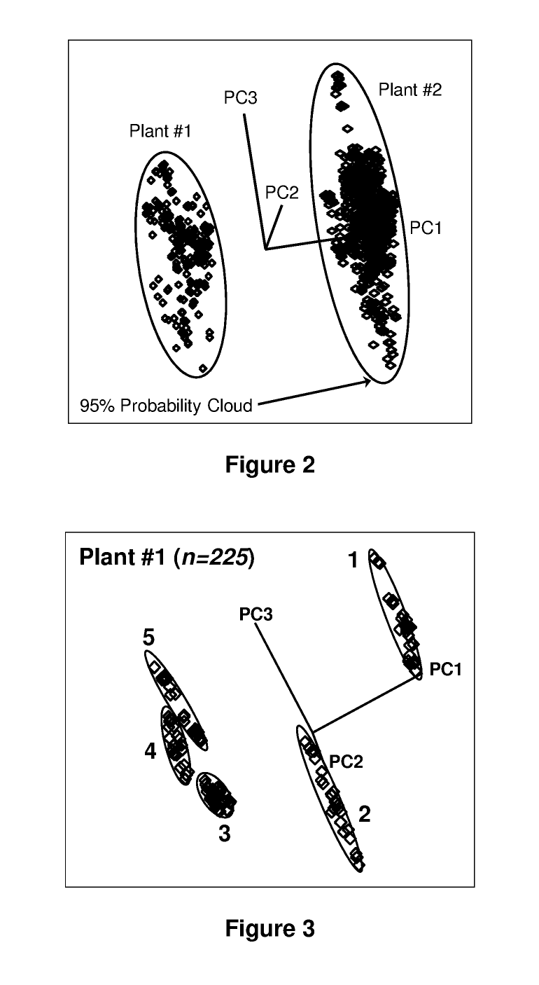

Many factors such as ingredients, milk composition, bacterial starter culture used for fermentation, etc. influence the final flavor of Cheddar cheese (Bachmann et al., 1999; Chen et al., 1998). Furthermore many plants develop their own strains of non-starter lactic acid bacteria with time, which significantly influence the final composition and flavor of the cheese. Our research showed marked differences in the chemical composition of samples from the two production plants (FIG. 2).

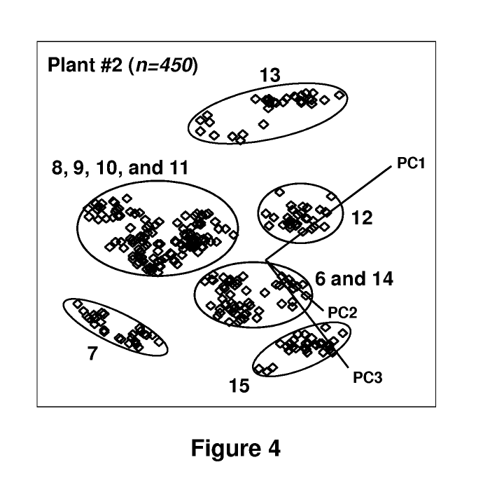

This apart from indicating the possibility of identifying the production plant based on the spectra also suggests that a separate flavor classification model is needed for each production plant. The discrimination of the 5 samples from Plant #1 is shown in FIG. 3.

All the five samples formed tight and distinct clusters. The distance between the clusters in a SIMCA plot is represented by the interclass distance (ICD). Greater the distance between two clusters the greater is the difference in composition of samples belonging to those clusters. As a rule of thumb, a distance of over 3 indicates that the samples are well separated and hence different (Kyalheim and Karstand, 1992).

Table 1. Interclass distances of Cheddar cheese samples from Plant #1. Greater the distance between two samples the greater is the difference between them.

TABLE 1
Interclass distances of Cheddar cheese samples
(Plant #1)

| Samples¹ | 1 | 2 | 3 | 4 | 5 |
|---|---|---|---|---|---|
| 1 | 0.00 | 4.75 | 6.14 | 29.62 | 12.49 |
| 2 | 4.75 | 0.00 | 3.70 | 31.56 | 13.99 |
| 3 | 6.14 | 3.70 | 0.00 | 28.21 | 13.96 |
| 4 | 29.62 | 31.56 | 28.21 | 0.00 | 15.85 |
| 5 | 12.49 | 13.99 | 13.96 | 15.85 | 0.00 |

¹Sample 1 - Slight Burn, Sample 2 - Sour, Sample 3 - Good, Sample 4 - Fermented, and Sample 5 - Unclean.

Table 1 shows the interclass distance values of the 5 samples from Plant #1. The samples with fermented flavor and unclean flavor, which are considered undesirable, clustered far from other samples. The ICD of fermented sample and unclean sample from good Cheddar were 28.21 and 13.96 units, respectively. Samples with slight burn and slight sour notes, which were not major flavor defects, clustered relatively close to the good Cheddar, with ICDs of 6.14 and 3.70, respectively.

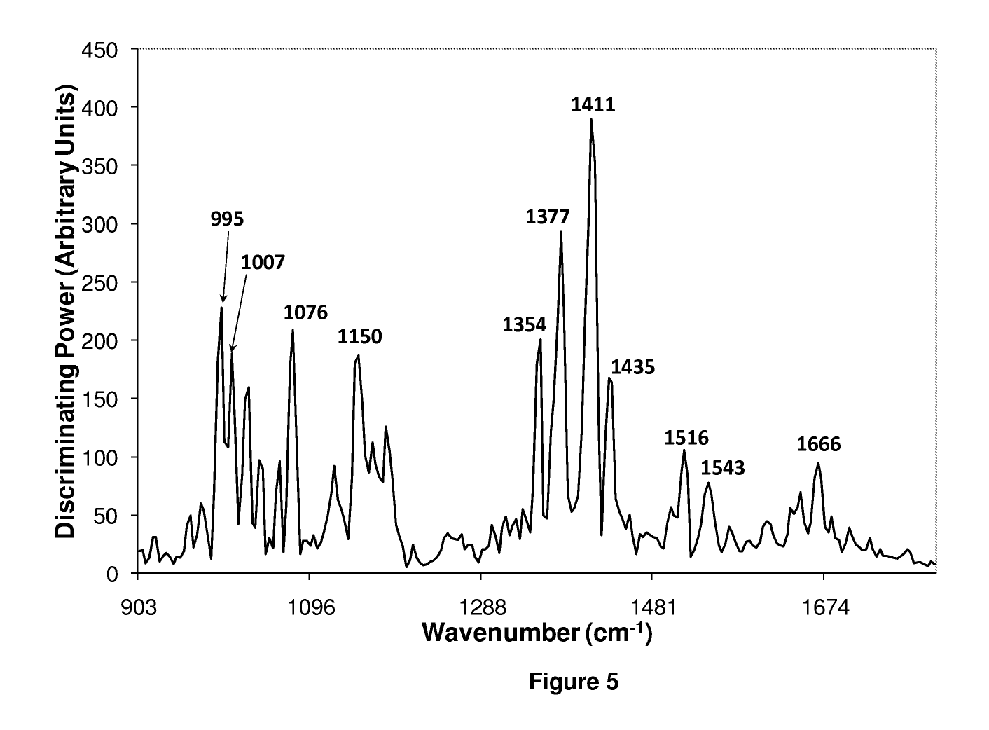

Samples from Plant #2 also exhibited very good clustering based on their flavor quality (FIG. 4). The ICDs for samples from Plant #2 are shown in Table 2.

Table 2. Interclass distances of Cheddar cheese samples from Plant #2. Greater the distance between two samples the greater is the difference between them.

TABLE 2
Interclass distances of Cheddar cheese samples (Plant #2)

| Samples | 6 | 7 | 8 | 9 | 10 | 11 | 12 | 13 | 14 | 15 |
|---|---|---|---|---|---|---|---|---|---|---|
| 6 | 0.00 | 5.08 | 3.81 | 5.58 | 5.42 | 3.97 | 5.39 | 8.48 | 2.99 | 5.51 |
| 7 | 5.08 | 0.00 | 6.02 | 7.70 | 7.06 | 7.18 | 9.79 | 12.56 | 4.63 | 7.49 |
| 8 | 3.81 | 6.02 | 0.00 | 2.66 | 1.96 | 1.39 | 5.62 | 8.71 | 3.58 | 8.89 |
| 9 | 5.58 | 7.70 | 2.66 | 0.00 | 2.23 | 2.79 | 6.06 | 8.58 | 3.98 | 10.37 |
| 10 | 5.42 | 7.06 | 1.96 | 2.23 | 0.00 | 1.80 | 6.35 | 8.73 | 3.62 | 10.29 |
| 11 | 3.97 | 7.18 | 1.39 | 2.79 | 1.80 | 0.00 | 5.17 | 8.74 | 3.25 | 7.94 |
| 12 | 5.39 | 9.79 | 5.62 | 6.06 | 6.35 | 5.17 | 0.00 | 5.61 | 6.29 | 6.49 |
| 13 | 8.48 | 12.56 | 8.71 | 8.58 | 8.73 | 8.74 | 5.61 | 0.00 | 8.87 | 12.07 |
| 14 | 2.99 | 4.63 | 3.58 | 3.98 | 3.62 | 3.25 | 6.29 | 8.87 | 0.00 | 7.82 |
| 15 | 5.51 | 7.49 | 8.89 | 10.37 | 10.29 | 7.94 | 6.49 | 12.07 | 7.82 | 0.00 |

Sample 6 and 14 - Good Cheddar,
Sample 7 - Fermented,
Sample 8, 9, 10, and 11 Sour Flavor, and
Sample 15 - Sulfide.

Samples with fermented flavor and low flavor clustered far from good Cheddar (ICD>3.0). Their ICDs from good Cheddar were 5.08 and 8.48, respectively. Samples with minor defects such as slight sour, slight acid, sulfide flavor and slight low flavor clustered close to the good Cheddar, with relatively low interclass distance. In flavor categories that contained more than one sample (Good Cheddar, and Sour and Slight Acid), samples within the flavor category clustered close to each other (ICD<3.0) and away from other categories (ICD>3.0). All the samples in the Sour and Slight Acid category (Samples 8, 9, 10, and 11) had ICD less than 3.0 among themselves and greater than 3.0 for samples outside the cluster (Table 2).

Similarly, the good Cheddar samples (Samples 6 and 14) had ICD of less than 3.0 between themselves and ICD greater than 3.0 from other clusters. These data clearly indicate that the developed extraction method combined with the FT-IR spectroscopy is useful for the analysis of, and ultimately for, quality control of Cheddar cheese flavor.

Identification of IR Bands Responsible for Classification

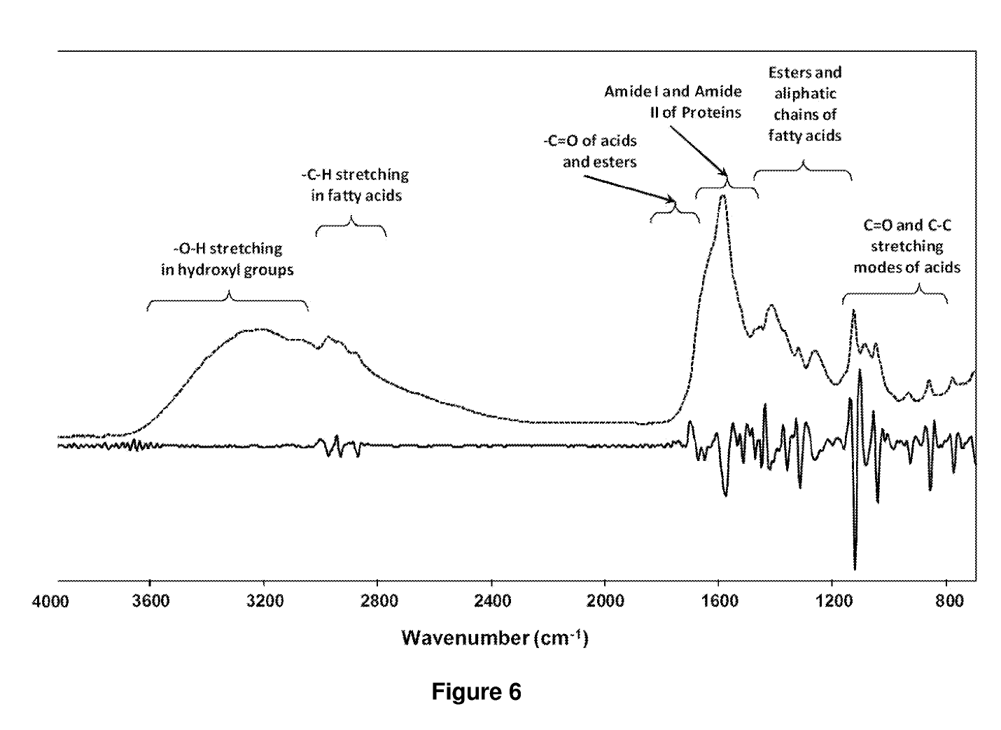

The spectral wavenumbers and the associated functional groups that were responsible for the classification of the cheeses in SIMCA plot can be identified using the discriminating power plot. In the discriminating power plot each wavenumber in the spectral range is plotted against its importance in discriminating the samples that are in the model. The higher the value of discriminating power, the greater is the influence of that wavenumber in classifying the samples. The spectral regions and the associated functional groups/compounds responsible for the differentiation of the Cheddar cheese samples are highlighted in FIG. 5.

The spectral range 1800 to 900 cm⁻¹ was found to be important in the analysis of cheese flavor by FT-IR. The major bands responsible for the classification were 1411, 1377 and 1354 cm⁻¹. These bands mainly include stretching vibrations of carboxyl groups, C—H bending vibrations of methyl groups and C-N stretching vibrations of amines (Coates, 2000; Guillen et al., 1997). Koca et al. (2007) associated 1412 cm⁻¹ with C—H symmetric bending vibrations of short and medium chain fatty acids. Some organic sulfates have also been reported to absorb in this region (Coates, 2000). Stretching vibrations of C—O groups appear in the region from 1200 to 1000 cm⁻¹ (Rodriguez-Saona et al., 2006). These signals can be attributed to C—O containing compounds including alcohols, organic acids, fatty acids, lactones, keto acids, etc. Carboxyl groups of acids also absorb at around 1435 cm⁻¹. Several other bands, 995 cm⁻¹ associated with C—H in-plane bends of aromatic compounds, 1543 and 1516 cm⁻¹ associated with aromatic nitrogen compounds, and 1666 cm⁻¹ associated with Amide I bands of peptides also influenced the discrimination of Cheddar cheese based on flavor.

In another aspect, there is described herein a reliable sample preparation method and FT-IR spectroscopy for analysis of Cheddar cheese flavor quality. Cheddar cheeses can be differentiated based on their flavor, using multivariate classification models. The total analysis time, including sample preparation time was less than 20 min per sample. The results indicate that this method can also be used for detection of flavor quality defects in Cheddar cheese. Furthermore, differences in the composition of cheese from different production plants can also be elucidated. The method described herein can save time and money for the cheese industry. The method described herein can also enable better quality control and rapid monitoring of ripening process to achieve cheese of desired flavor quality.

### Example II

Multiple methods are required for analysis of cheese flavor quality and composition. Chromatography and sensory analyses are accurate but laborious, expensive and time consuming. A rapid and easy-to-use instrumental method based on Fourier transform infrared (FTIR) spectroscopy was developed for simultaneous analysis of Cheddar cheese composition and flavor quality. Twelve different Cheddar cheese samples ripened for 67 days were obtained from a commercial cheese manufacturer along with their moisture, pH, salt, fat content and sensory flavor quality data. Water soluble components were extracted from the cheese, dried on zinc selenide FTIR crystal and scanned (4000-700 cm⁻¹). Infrared spectra of the samples were correlated with their composition and flavor quality data to develop multivariate statistical regression and classification models. The models were validated using an independent set of ten 67-day old test samples. The infrared spectra of the samples were well defined, highly consistent within each sample and distinct from other samples. The regression models showed excellent fit (r-value>0.92) and can accurately determine moisture, pH, salt, and fat contents as well as the flavor quality rating in less than 20 min. Furthermore, cheeses can also be classified based on their flavor quality (slight acid, whey taint, good cheddar, etc.). The discrimination of the samples was due to organic acids, amino acids and short chain fatty acids (1800 to 900 cm⁻¹), which are known to contribute significantly to cheese flavor. The results show that the method described herein is a rapid, inexpensive, and reliable method for predicting composition and flavor quality of cheese.

Introduction

Flavor analysis using infrared spectroscopy is complicated due to difficulties in sampling procedures and interference from matrix compounds. Our previous research demonstrated a novel sample preparation procedure that enabled flavor quality analysis by FTIR spectroscopy (Subramanian and Rodriguez-Saona 2008b; Subramanian and others 2008).

Specific flavor notes can be correlated to the infrared spectra and cheese can be classified based on their flavor quality. In this paper we present the capability of an easy-to-use sample preparation method and FTIR spectroscopy to simultaneously determine pH, fat, moisture, salt, and flavor quality of Cheddar cheese.

Materials and Methods for Example II

Cheddar Cheese Samples

Twelve different Cheddar cheese samples (4 different production days and 3 different vats per production day) ripened for a period of 67 days were provided by a commercial cheese manufacturer. Moisture, fat, salt content and pH of the samples were determined by the manufacturer using standard methods. The flavor quality of the samples were rated on a scale of 1 to 10 (10 being the highest flavor quality rating) by trained quality assurance personnel in the production facility and provided along with the samples. Upon reception the samples were stored at −40° C. until analysis.

Sample Preparation and FTIR Analysis

Samples were prepared for FTIR analysis following the method described by Subramanian and others (2008). Powders of the cheese samples were prepared by cryogenically grinding (with liquid nitrogen) 20 g of the cheese. Exactly 0.1 g of the powder was mixed with 0.5 mL of water and sonicated (Fisher Scientific, Pittsburgh, Pa.) for 10 seconds. The water soluble fraction from the mixture was partitioned by adding 0.5 mL of chloroform and centrifuging (13000 rpm, 3 min). To 200 μL of the supernatant, equal volume of ethanol was added and centrifuged (13000 rpm, 3 min). The resulting supernatant was used for spectral measurements in a Varian 3100 FTIR spectrometer (Varian Inc., Palo Alto, Calif.). The spectrometer was equipped with a PERMAGLOW™ mid-IR source (4000-700 cm⁻¹), potassium bromated beam splitter and a deuterated triglycine sulfate detector. An infrared-transparent 3-bounce zinc selenide attenuated total reflectance (ATR) accessory (MIRacle™, Pike Technologies, Madison, Wis.) was used as sample holder. Exactly 7.5 μL of the extract was vacuum-dried on the crystal to form a film and scanned in the spectrometer in the mid-infrared region. Each spectrum was recorded by co-adding 64 scans, which theoretically yields a high signal-to-noise ratio of 8:1. Five independent extractions were performed for each sample and 3 spectra were collected for each extract, yielding at least 15 spectra per sample and a total of 180 spectra in the model. For the validation set, 10 samples were evaluated with a total of 150 spectra.

Multivariate Analyses

The classification and regression analyses of the spectral data were performed using Pirouette® (version 3.11, Infometrix Inc., Woodville, Wash.). The spectra of the cheese samples were mean-centered, derivatized (Savitzky-Golay polynomial filter with a 5-point window) and normalized prior to multivariate analysis.

The spectra were then matched with the pH, composition (moisture, salt, and fat) and flavor quality rating data to develop prediction models based on partial least squares regression (PLSR). A nonlinear iterative partial least-squares (NIPALS) algorithm was employed. Classification model to differentiate cheese based on their flavor was developed using soft independent modeling of class analogy (SIMCA). The data were projected onto the first three principal component axes to visualize clustering of samples in 3D space based on their flavor note (whey taint, slight acid, good cheddar, etc.). The spectral regions influencing the classification of the cheeses were determined from the measure of variable importance (discriminating power). The developed prediction models were validated with ten 67-days old independent test samples.

Results And Discussion For Example II

Extraction with organic solvents enabled removal of compounds that interfered with the detection of essential compounds such as organic acids and amino acids that contribute to the flavor (Subramanian and others 2008). Drying of the extract on the crystal resulted in the formation of a uniform film of sample. The drying time per sample was 3 min. FTIR spectra of the extracts were collected in the mid-infrared region (4000-700 cm⁻¹), using a 3-bounce zinc selenide ATR crystal. In a 3-bounce ATR-FTIR crystal, infrared light bounces on the sample 3 times, increasing the absorbance and hence the signal. The sample preparation method allowed for the collection of high-quality spectra with distinct spectral features that were very consistent within each sample.

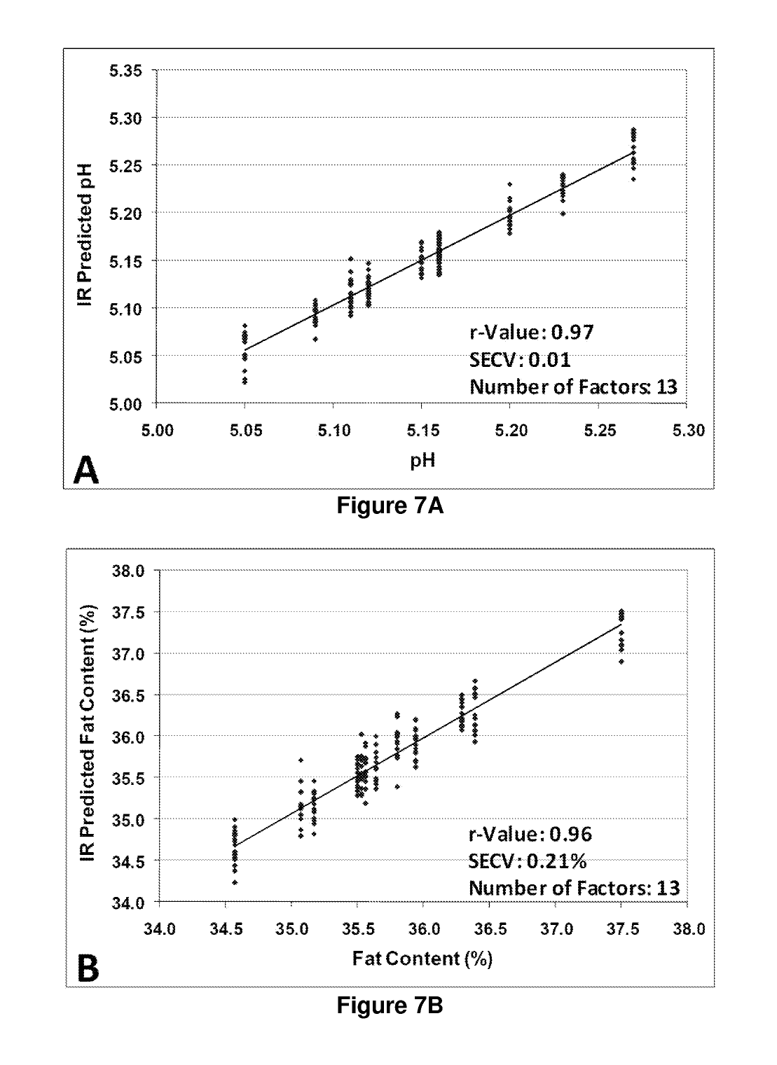

The FTIR spectra reflect the total chemical composition of the cheese extract, with absorbance bands due to acids, esters, alcohols and peptides. The band intensities vary with the overall concentration of the chemical functional groups in the sample. The raw spectra were transformed into their second derivatives to remove the baseline shifts, improve band resolution, and reduce noise and variability between replicates (Kansiz and others 1999). A typical FTIR spectrum of Cheddar cheese extract and its second derivative are shown in FIG. 6. The region from 4000 to 3100 cm⁻¹ consists of absorbance from O—H and N—H stretching vibrations of hydroxyl groups and Amide A of polypeptides and amino acids, respectively. The C—H stretching vibrations of —CH3 and >CH2 functional groups of fatty acids appear between 3100 and 2800 cm⁻¹. The spectral range 1800 to 900 cm⁻¹ contains signals from polypeptides, amino acids, carbonyl groups of fatty acids, hydroxyl groups, carboxylic acid groups and fatty acid esters (typically short chain). Visual comparison of the raw spectra showed numerous differences between cheeses, especially in the spectral region 1800-900 cm⁻¹.

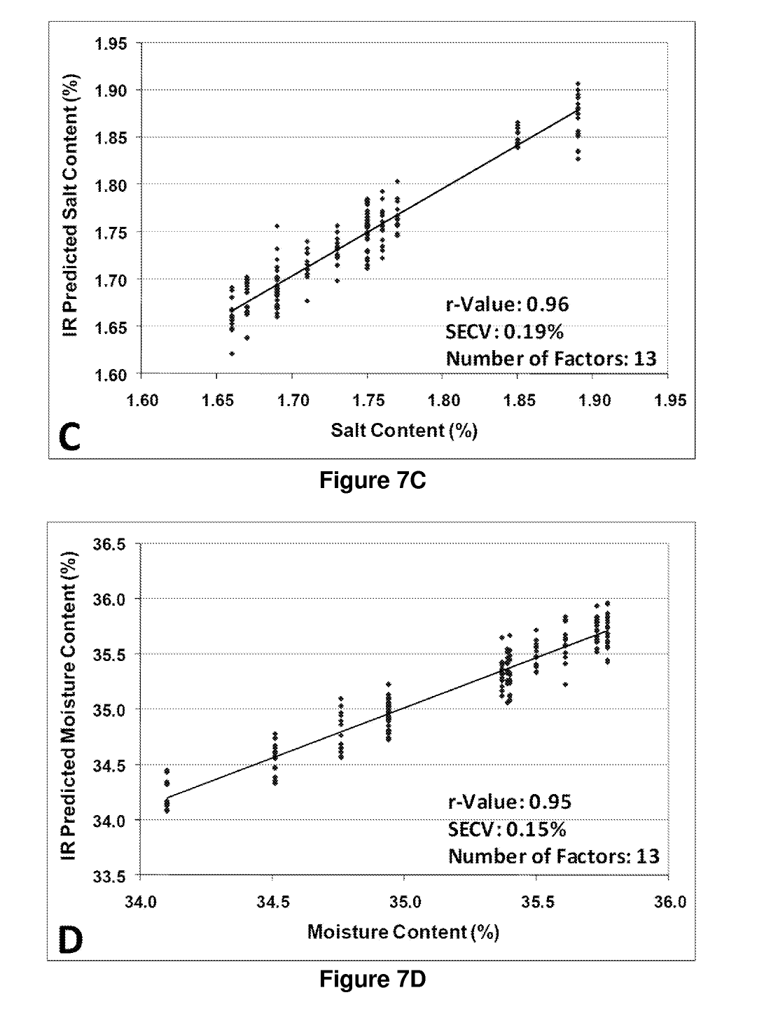

The transformed spectra were correlated with the composition (fat, salt, and moisture), pH and quality rating and analyzed by partial least squares regression (PLSR) with cross-validation (leave-one-out approach) to generate calibration models. PLSR is a bi-linear regression analysis which determines the analyte's concentration (Y-variable) by regressing a small number of orthogonal factors that are linear combinations of variables (X-variable; infrared wavenumbers). These orthogonal factors, also called latent variables, explain as much covariance as possible between X and Y (Bjorsvik and Martens 1992). PLSR provides information-rich data set of reduced dimensionality, good reproducibility and lesser noise and has been very successful in developing calibration models for spectroscopic data (Martens and Martens 2001). The PLSR calibration models for pH, fat, salt, moisture and flavor quality score are shown in FIG. 7. All the five models exhibited excellent correlation with coefficient of correlation (r) values greater than 0.92. The pH of the cheese can be predicted with a standard error of cross-validation (SECV) value of just 0.01 (FIG. 7A). The SECV is an estimate of the error expected when independent samples are predicted using the model. Similarly, the models for predicting fat content, salt, and moisture of the cheese samples exhibited very low SECV values of 0.21% (FIG. 7B), 0.19% (FIG. 7C), and 0.15% (FIG. 7D), respectively. The flavor quality scores and the IR spectra also exhibited very high correlation (FIG. 7E) with a coefficient of correlation of 0.92 and SECV of 0.39. This indicates that it is possible to predict flavor quality score within an error of just 0.39.

Until now, the industry had to determine the composition and pH require the use of multiple techniques and several organic chemicals. Furthermore, these techniques were complicated and expensive.

The PLSR prediction models described above were validated with an independent set of ten 67-days old test samples. The pH, fat, salt, moisture content and the flavor quality score were predicted using the developed models. The values predicted using the models and the actual values are presented in Table 3.

TABLE 3
Predicted and actual pH, fat, moisture, salt and flavor quality score for the 67-day old
test samples.

| | | | | | | | | | Flavor Quality | |
|---|---|---|---|---|---|---|---|---|---|---|
| | pH | | Fat (%) | | Moisture (%) | | Salt (%) | | Score | |
| Test | Predicted | Actual | Predicted | Actual | Predicted | Actual | Predicted | Actual | Predicted | Actual |
| A | 5.18 | 5.18 | 34.79 | 34.80 | 35.77 | 35.80 | 1.83 | 1.86 | 6.02 | 6.00 |
| B | 5.13 | 5.12 | 35.03 | 34.40 | 35.65 | 36.20 | 1.82 | 1.84 | 6.17 | 6.00 |
| C | 5.10 | 5.11 | 35.28 | 35.40 | 35.45 | 35.50 | 1.77 | 1.78 | 6.93 | 7.00 |
| D | 5.14 | 5.17 | 35.20 | 35.10 | 35.59 | 35.60 | 1.79 | 1.83 | 7.49 | 8.00 |
| E | 5.11 | 5.11 | 35.19 | 35.50 | 35.71 | 35.50 | 1.80 | 1.78 | 7.36 | 7.00 |
| F | 5.08 | 5.07 | 35.07 | 35.20 | 35.78 | 35.60 | 1.78 | 1.73 | 8.81 | 9.00 |
| G | 5.11 | 5.10 | 35.07 | 35.20 | 35.47 | 35.40 | 1.76 | 1.75 | 7.65 | 8.00 |
| H | 5.13 | 5.10 | 35.06 | 35.00 | 35.86 | 35.80 | 1.80 | 1.78 | 7.75 | 8.00 |
| I | 5.20 | 5.19 | 35.17 | 35.40 | 35.86 | 35.60 | 1.76 | 1.74 | 6.20 | 6.00 |
| J | 5.23 | 5.22 | 34.88 | 34.60 | 36.23 | 36.50 | 1.81 | 1.81 | 4.88 | 5.00 |

The models showed excellent predictive capability. The average percentage deviation of predicted values from actual values were 0.23, 0.57, 0.47, 1.23, and 3.1 for pH, fat, moisture, salt and flavor quality score, respectively. These data clearly indicate the reliability and the usefulness of the method described herein for simultaneous analysis of different cheese characteristics.

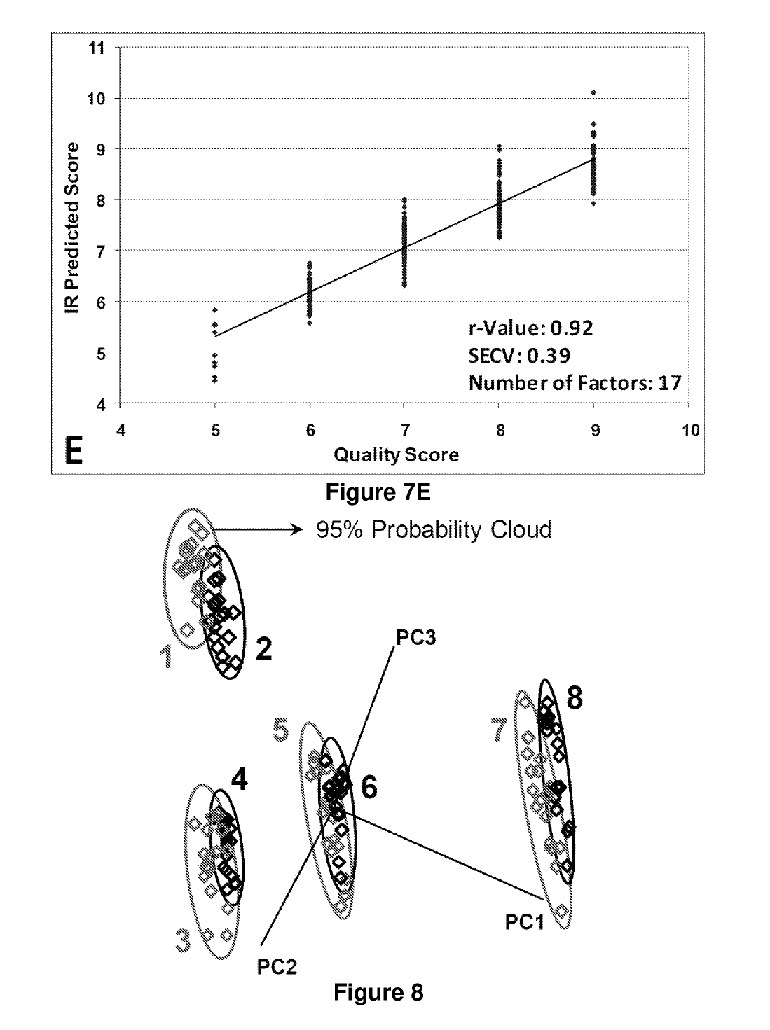

The spectra were also analyzed the soft independent modeling of class analogy (SIMCA) with the aim of classifying the cheese based on their flavor notes. SIMCA is a multivariate statistical technique based on principal component analysis (PCA) that reduces the dimensionality of multivariate data sets. In SIMCA, training sets are assigned to classes and a principle component model is generated for each class with distinct confidence regions within them (De Maesschalck and others 1999). A scores plot is constructed by projecting the actual data on the first three principal components that explain the most variance in the training data set. The variance explained by the class model describes the signal and the residual variance describes the noise in the data set and is used to define probability boundaries around the class. Comparison of the average residual variance of samples in a class and the residual variance of an unknown sample can help in identification of the unknown sample (Lavine 2000). The performance of this method depends not only on the difference between classes, but also strongly on the training set for each class (Candofi and others 1999). The SIMCA classification plot for discrimination of cheese based on flavor is shown in FIG. 8 (only 8 of the 12 samples are shown for ease of visualization). All the eight samples formed tight clusters and the location of the clusters in 3D space correlated well with their flavor quality. The good samples clustered together (cluster 5 and 6) and away from the samples with defects. Clusters with defects in flavor notes clustered away from the “good creamy cheddar” but close to samples with similar flavor note.

The distance between the clusters in a SIMCA plot is represented by the interclass distance (ICD). It is a measure of the quality of the data. Greater the distance between two clusters the greater is the difference in composition of samples belonging to those clusters. As a rule of thumb, a distance of over 3 indicates that the samples are well separated (Kyalheim and Karstand 1992). Samples that had similar flavor quality had ICD of less than 3 within themselves and an ICD of greater than 3 when compared to samples with a different flavor quality (data not shown). This data supports our previous publication on the possibility of classifying cheese samples based on flavor notes (Subramanian and others 2008).

The spectral wavenumbers and the associated functional groups that were responsible for the classification of the cheeses in SIMCA plot can be identified using the discriminating power plot. In the discriminating power plot each wavenumber in the spectral range is plotted against its importance in discriminating the samples that are in the model. The higher the value of discriminating power, the greater is the influence of that wavenumber in classifying the samples. The discriminating power also indicates the quality of the data and variables with low discriminating power are usually deleted because they contribute only to noise in the data set (Lavine 2000). The spectral regions and the associated functional groups/compounds responsible for the differentiation of the Cheddar cheese samples are highlighted in FIG. 8. The spectral range 1800-900 cm⁻¹ was found to be important in the analysis of cheese flavor by FTIR. This region consists of signals from C—O and C═O (˜1175 cm⁻¹), C—H bending (˜1450 cm⁻¹), esters (1750-1700 cm⁻¹) and C—O stretching (˜1240 and 1170 to 1115 cm⁻¹) (Rodriguez-saona and others 2006). In the case of Cheddar cheese extract compounds containing these functional groups include the organic acids, alcohols, short chain fatty acids and their esters, amino acids and small water soluble peptides, all of which are important for flavor.

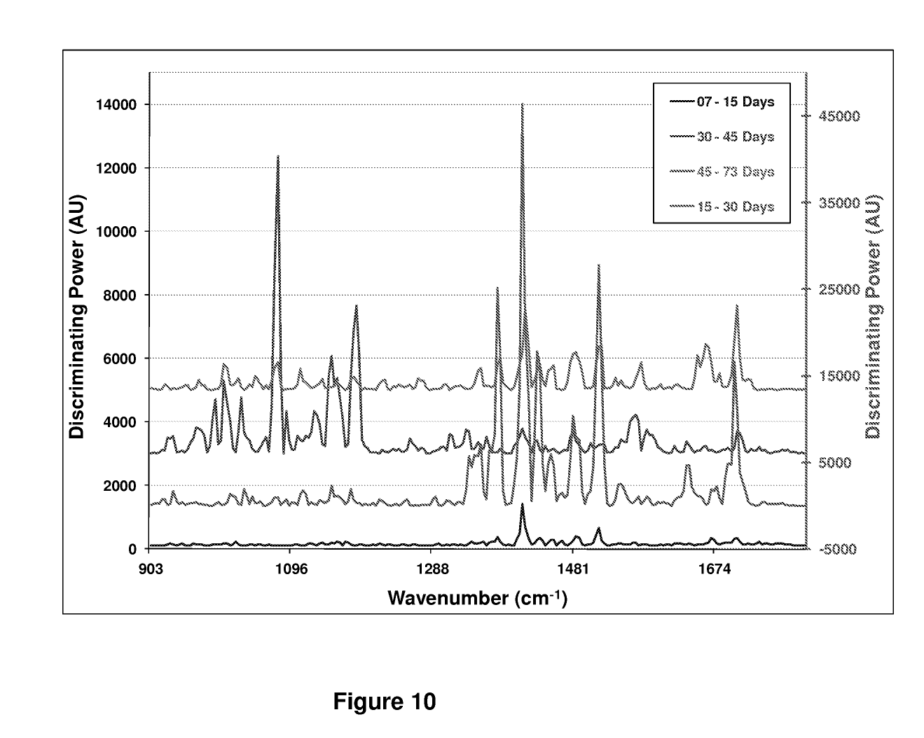

Apart from determination of age, composition and flavor quality of the cheeses, this technique also enabled monitoring some of the biochemical reactions that took place during cheese ripening. For example, the changes that occurred during various stages of ripening in a sample with a final flavor quality of “Good Creamy Cheddar” are shown in FIG. 10. Between day 7 and day 15, minor changes occurred in fatty acid and amino acid composition, which may represent initial stages of fat and protein breakdown. The period from day 15 to day 30 exhibited the greatest amount of changes possibly due to heightened protein and fat breakdown (1550-1300 cm⁻¹ and 1710 cm⁻¹). The third stage (day 30-day 45) shows the appearance of breakdown products, especially organic acids (1200-900 cm⁻¹). The final stage (day 45-day 73) of Cheddar cheese ripening showed relatively less intense but numerous changes in the regions that correspond to amino acids, short chain fatty acids and organic acids, which signifies formation of flavor related minor compounds. Thus, the described FT-IR method provides valuable information on flavor related biochemical changes during ripening. Such information is useful in understanding flavor formation during cheese ripening as well as monitoring and controlling cheese ripening process to achieve desired flavor formation.

A rapid, easy-to-use and reliable FTIR technique was developed for simultaneous analysis of Cheddar cheese composition and flavor quality. The fat, salt, moisture, pH and flavor quality score of the cheese can be predicted in less than 20 min. Additionally, the infrared spectra correlated well with the flavor notes. This method provides a rapid quality control method for the cheese industry to utilize in providing more consistency to cheese analysis and grading and improving quality.

Application of this method for Swiss cheese analysis yielded similar results. Thus, it to be understood that the method described herein is useful, not only with Cheddar cheese, but also with other types of cheese.

### Example III

The extraction method is also useful for rapid cheese quality and composition analysis, significantly reducing the time and effort compared to prior techniques.

Cheese samples were classified based on their flavor quality descriptors (fermented, unclean, low flavor, sour, good cheddar, etc.). This method provides a rapid, inexpensive, high-throughput and easy-to-use method to the cheese industry for predicting the flavor quality of cheese. In addition, this method saves time and money for the cheese industry. Further, the method described herein enables better quality control and rapid monitoring of ripening processes, thereby achieving cheese of desired flavor quality.

The method described herein is also useful to classify Cheddar cheese based on flavor and extends its application to prediction of age, pH, moisture, salt and fat contents and monitoring the biochemical changes in cheese during the ripening process. The age, fat, salt, moisture, pH and flavor quality of the cheese can be predicted in less than about 20 min. Furthermore, this method also provides useful data on the changes occurring during cheese ripening process.

In another aspect, the method described herein is useful to predict the flavor quality of Cheddar cheese early in the ripening process. This method has both applied and scientific significance. Predicting the flavor of cheese early in the ripening process can: 1) enable manufacturers to identify cheese that might develop undesirable flavor and modify the ripening process parameters to aid desirable flavor formation; 2) save money by eliminating ripening costs for cheese that have developed undesirable flavor; and, 3) help in making decisions on the possible applications of the cheese based on its flavor

Materials And Methods For Example III

Cheese Samples

Two sets of Cheddar cheese samples were obtained. The first set consisted of 17 different cheddar cheeses ripened for a period of 67 days. Samples were collected in on days 30 and 67 of ripening. These samples were extracted and analyzed to develop models to predict the flavor quality of the cheese early in the ripening process as well as the composition. The second set consisted for 10 test samples to validate the developed models. These samples were also ripened for 67 days and sampled on days 30 and 67. The composition and flavor quality scores for these samples were provided by the manufacturer, for use as a comparison.

Sample Preparation and FT-IR Analysis

The samples were extracted using water, chloroform and ethanol by following the procedure described herein. The prepared extracts were analyzed using FT-IR spectroscopy.

Multivariate Analysis

The spectra from the first set of samples (17) were correlated with composition data provided by the manufacturer to develop partial least squares prediction models to predict pH, salt, moisture and fat contents. Additionally another prediction model was built correlating spectra of 30-day old cheese samples with their flavor quality rating at the end of 67-days. This was done to investigate the possibility of predict an expected final flavor quality of cheese early in the ripening process (at 30 days of age). The performances of these developed models were validated using 10 test samples. The composition of test samples and the expected final flavor quality of the samples were predicted using the models and compared with the actual values supplied by the manufacturer.

Results And Discussion For Example III

Table 4 summarizes the triplicate average predicted and actual pH, fat, moisture and salt contents. The spectra of the 30-day old test cheeses were used for the predictions. Excellent accuracy of prediction was observed. The data in Table 4 further show the usefulness of the method described herein to predict the cheese composition both rapidly and accurately.

TABLE 4
Predicted and Actual pH, Fat, Moisture and Salt content of the cheese samples.

| | | | | | | | Moisture | |
|---|---|---|---|---|---|---|---|---|
| Sample | pH | | Fat (%) | | (%) | | Salt (%) | |
| Code | Predicted | Actual | Predicted | Actual | Predicted | Actual | Predicted | Actual |
| TA30 | 5.18 | 5.18 | 34.79 | 34.80 | 35.77 | 35.80 | 1.83 | 1.86 |
| TB30 | 5.13 | 5.12 | 35.03 | 34.40 | 35.65 | 36.20 | 1.82 | 1.84 |
| TC30 | 5.10 | 5.11 | 35.28 | 35.40 | 35.45 | 35.50 | 1.77 | 1.78 |
| TD30 | 5.14 | 5.17 | 35.20 | 35.10 | 35.59 | 35.60 | 1.79 | 1.83 |
| TE30 | 5.11 | 5.11 | 35.19 | 35.50 | 35.71 | 35.50 | 1.80 | 1.78 |
| TF30 | 5.08 | 5.07 | 35.07 | 35.20 | 35.78 | 35.60 | 1.78 | 1.73 |
| TG30 | 5.11 | 5.10 | 35.07 | 35.20 | 35.47 | 35.40 | 1.76 | 1.75 |
| TH30 | 5.13 | 5.10 | 35.06 | 35.00 | 35.86 | 35.80 | 1.80 | 1.78 |
| TI30 | 5.20 | 5.19 | 35.17 | 35.40 | 35.86 | 35.60 | 1.76 | 1.74 |
| TJ30 | 5.23 | 5.22 | 34.88 | 34.60 | 36.23 | 36.50 | 1.81 | 1.81 |

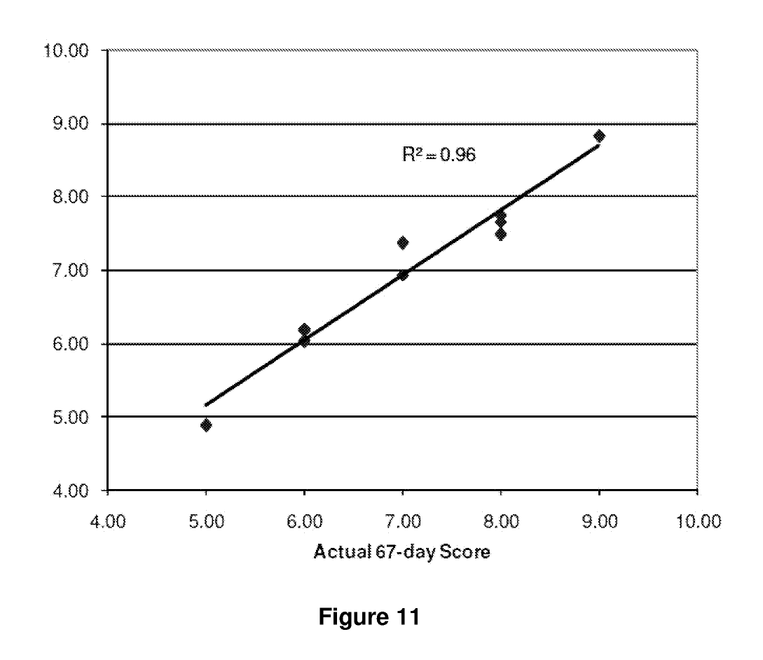

Table 5 summarizes the triplicate average predicted and actual flavor quality scores. The spectra of the 30-day old test cheeses were used to predict the expected flavor quality score at the end of 67 days. The prediction models showed excellent accuracy. The correlation between the predicted score and the actual scores is shown in FIG. 11. The coefficient of determination (R²) value was as high as 0.96 and clearly shows one use of this method to predict the flavor quality early in the ripening process.

TABLE 5
Predicted and Actual flavor quality score of the cheese samples.

67-day Flavor Quality Score

| Sample Code | Predicted | Actual |
|---|---|---|
| TA30 | 6.02 | 6.00 |
| TB30 | 6.17 | 6.00 |
| TC30 | 6.93 | 7.00 |
| TD30 | 7.49 | 8.00 |
| TE30 | 7.36 | 7.00 |
| TF30 | 8.81 | 9.00 |
| TG30 | 7.65 | 8.00 |
| TH30 | 7.75 | 8.00 |
| TI30 | 6.20 | 6.00 |
| TJ30 | 4.88 | 5.00 |

This example demonstrates the application of the novel extraction method to predict the expected flavor quality scores of cheddar cheese early in the ripening process. The data described herein illustrate the usefulness of this method to simultaneously determine the flavor quality, composition, and expected final flavor quality of Cheddar cheese in less than 20 min. This method provides a benefit with the industry as well as cheese research for the currently multiple methods, extensive labor, excessive use of organic solvents, and complicated methodologies to determine flavor quality and composition.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

Adams M J, Latham K, Barnett N W, Poynton A J. 1999. Calibration models for determining moisture and fat content of processed cheese using near-infrared spectrometry. J. Sci. Food Agric. 79:1232-6.
Adda, J., Gripon, J. C., and L. Vassal. 1982. The chemistry of flavor and texture generation in cheese. Food Chem. 9:115-129.
Akalin, A. S., Gonc, S., and Y. Akba. 2002. Variation in organic acids content during ripening of pickled white cheese. J. Dairy Sci. 85:1670-1676.
Alli, I., Okoniewska, M., Gibbs, B. F., and Y. Konishi. 1998. Identification of peptides in Cheddar cheese by electrospray ionization mass spectrometry. Int. Dairy J. 8:643-649.
Aston, J. and L. K. Creamer. 1986. Contribution of the components of water-soluble fraction to the flavor Cheddar cheese. New Zealand Dairy Sci. Tech. 21:229-248.
Bachmann, H P., Butikofer, U., and J. Meyer. 1999. Prediction of flavor and texture development in Swiss-type cheeses. Lebensm.-Wiss. U.-Technol. 32:284-289.
Bjorsvik H R, Martens H. 1992. Data analysis: calibration of NIR instruments by PLS regression. In: Burns D A, Ciurczak E W, editors. Handbook of Near-Infrared Analysis. New York: Taylor and Francis. p159-80.
Blazquez C, Downey G, O'Callaghan D, Howard V, Delahunty C, Sheehan E, Everard C, O'Donnell C P. 2006. Modelling of sensory and instrumental texture parameters in processed cheese by near infrared reflectance spectroscopy. J. Dairy Res. 73:58-69.
Candofi A, De Maesschalck R, Massart D L, Hailey P A, Harrington A C E. 1999. Identification of pharmaceutical excipients using NIR spectroscopy and SIMCA. J. Pharma. Biomed. Anal. 19:923-35.
Cattaneo, T. M. P., Giardina, C., Sinelli, N., Riva, M., and R. Giangiacomo. 2005. Application of FT-NIR and FT-IR spectroscopy to study the shelf-life of Crescenza cheese. Int. Dairy J. 15:693-700.
Chen, M., and J. Irudayaraj. 1998. Sampling technique for cheese analysis by FTIR spectroscopy. J. Food Sci. 63:96-99.
Chen, M., Irudayaraj, J., and D. J. McMahon. 1998. Examination of full fat and reduced fat Cheddar cheese during ripening by Fourier transform infrared spectroscopy. J. Dairy Sci. 81:2791-2797.
Coates, J. 2000. Interpretation of Infrared Spectra, a Practical Approach. In Encyclopedia of Analytical Chemistry, Meyers R R ed. pp. 10815-10837. John Wiley and Sons LTD, Chichester.
De Maesschalck R, Candofi A, Masart D L, Heuerding S. 1999. Decision criteria for soft independent modeling of class analogy applied to near infrared data. Chemometr. Intell. Lab. Syst. 47:65-77.
Downey G, Sheehan E, Delahunty C, O'Callaghan D, Guinee T, Howard V. 2005. Prediction of maturity and sensory attributes of Cheddar cheese using near-infrared spectroscopy. Int. Dairy J. 15:701-9.
Engel, E., J. B. Lombardot, A. Garem, N. Leconte, C. Septier, J. L. Le Quere and C. Salles. 2002. Fractionation of the water-soluble extract of cheese made from goat's milk by filtration methods: behavior of fat and volatile compounds. Int. Dairy J. 12:609-619.
Engels, W. J. M., R. Dekker, C. de Jong, R. Neeter and S. Visser. 1997. A comparative study of volatiles compounds in the water-soluble fraction of various types of ripened cheese. Int. Dairy J. 7:255-263.
Farkye, N. Y., and P. F. Fox. 1990. Objective indices of cheese ripening. Trends Food Sci. Technol. 1:37-40.
Guillen, M. D., and N. Cabo. 1997. Infrared spectroscopy in the study of edible oils and fats. J. Sci. Food Agric. 75:1-11.
Kansiz, M., Heraud, P., Wood, B., Burden, F., Beardall, J., and D. McNaughton. 1999. Fourier transform infrared microspectroscopy and chemometrics as a tool for the discrimination of cyanobacterial strains. Phytochemistry. 52, 407-417.
Karoui, R., Dufour, E., Pillonel, L., Picque, D., Cattenoz, T., and J. O. Bosset. 2004. Determining the geographic origin of Emmental cheeses produced during winter and summer using a technique based on concatenation of MIR and fluorescence spectroscopic data. Eur. Food Res. Technol. 219:184-189.
Karoui R, Dufour E, Pillonel L, Schaller E, Picque D, Cattenoz T, Bosset J O. 2005. The potential of combined infrared and fluorescence spectroscopies as a method of determination of geographic origin of Emmental cheeses. Int. Dairy J. 15:287-98.
Koca, N., Rodriguez-Saona, L. E., Harper, W. J., and V. B. Alvarez. 2007. Application of Fourier transform infrared (FTIR) spectroscopy for monitoring short-chain free fatty acids in Swiss cheese. J. Dairy Sci. 90:3596-603.
Kosikowski, F. V., and G. Mocquot. 1958. Advances in cheese technology. FAO Agric. Stud. Nr. 38, Rome, Italy: Food and Agriculture Organization (FAO). 15 p.
Kvalheim, O. M., and T. V. Karstand. 1992. SIMCA—classification by means of disjoint cross validated principal components models. In: Brereton, R. G. (Eds.), Multivariate pattern recognition in chemometrics: illustrated by case studies. Elsevier, Amsterdam, Netherlands, pp 209-248.
Lavine B K. 2000. Clustering and classification of analytical data. In: Meyers R A, editor. Encyclopedia of Analytical chemistry: instrumentation and applications. New York: John Wiley and Sons Ltd. p 9689-710.
Lefier D, Lamprell H, Mazerolles G. 2000. Evolution of Lactococus strains during ripening in Brie cheese using Fourier transform infrared spectroscopy. Lait. 80:247-54.
Lues, J. F. R., and A. C. M. Bekker. 2002. Mathematical expression for organic acids in early ripening of a Cheddar cheese. J. Food Compos. Anal. 15:11-17.
Mark, H. 2001. Multilinear regression and principal component analysis. In: Burns, D. A., Ciurczak, E. W. (Eds.), Handbook of Near-Infrared Analysis. Taylor and Francis, N.Y., pp. 129-184.
Marten H, Martens M. 2001. Multivariate analysis of quality: an introduction. New York: John Wiley and Sons Ltd. p 445.
Mazerrolles G, Devaux M F, Duboz G, Duployer M H, Riou N M, Dufour E. 2001. Infrared and fluorescence spectroscopy for monitoring protein structure and interaction changes during cheese ripening. Lait. 81:509-27.
McQueen, D. H., Wilson, R., Kinnunen, A., and E. P. Jensen. 1995. Comparison of two infrared spectroscopic methods for cheese analysis. Talanta. 42:2007-2015.
McSweeney, P. L. H., and M. J. Sousa. 2000. Biochemical pathways for the production of flavour compounds in cheeses during ripening: A review. Lait. 80, 293-324.
National Agricultural Statistics Service, United States Department of Agriculture, www.nass.usda.gov, Accessed on Mar. 31, 2007.
Partidario, A. M., Barbosa, M., and L. V. Boas. 1998. Free fatty acids, triglycerides and volatile compounds in Serra da Estrela cheese-changes throughout ripening. Int. Dairy J. 8:873-881.
Pillonel L, Luginbühl W, Picque D, Schaller E, Tabacchi R, Bosset J O. 2003. Analytical methods for the determination of the geographic origin of Emmental cheese: mid- and near-infrared spectroscopy. Eur. Food Res. Technol. 216:174-8.
Rodriguez-Otero J L, Hermida M, Cepeda A. 1995. Determination of fat, protein, and total solids in cheese by near-infrared reflectance spectroscopy. J. AOAC Int. 78:802-6.
Rodriguez-saona, L. E., Koca, N., Harper, W. J., and V. B. Alvarez. 2006. Rapid determination of Swiss cheese composition by Fourier transform infrared/attenuated total reflectance spectroscopy. J. Dairy Sci. 89:1407-1412.
Seitz, E. W. 1990. Microbial- and enzyme-induced flavor in dairy foods. J. Dairy Sci. 73:3663-3691.
Singh, T K., Drake, M. A., and K. R. Cadwallader. 2003. Flavor of Cheddar cheese: A chemical and sensory perspective. Comp. Rev. Food Sci. Food Saf. 2: 166-189.
Skeie S, Feten G, Almøy T, Østlie H, Isaksson T. 2006. The use of near infrared spectroscopy to predict selected free amino acids during cheese ripening. Int. Dairy J. 16:236-42.
Sørensen L, Jepsen R. 1998. Assessment of sensory properties of cheese by near-infrared spectroscopy. Int. Dairy J. 8:863-71.
Subramanian A, Rodriguez-Saona L E. 2008a. Fourier transform infrared (FT-IR) spectroscopy. In: Sun D W, editor. Infrared Spectroscopy for Food Quality Analysis and Control. Dublin, Ireland: Elsevier. p 150-184. Forthcoming.
Thierry, A., Maillard, M. B., and J. L. Le Quere. 1999. Dynamic headspace analysis of Emmental aqueous phase as a method to quantify changes in volatile flavor compounds during ripening. Int. Dairy. J. 9:453-463.
Urbach, G. 1993. Relations between cheese flavor and chemical composition. Int. Dairy J. 3:389-422.

What is claimed is:

1. A method for evaluating a sample cheese comprising:
determining one or more infra-red (IR) spectra of the cheese at least one point in time by using a rapid solvent extraction method in combination with Fourier transform infrared (FT-IR) spectroscopy.

2. The method of claim 1, wherein the sample cheese is classified based on flavor quality.

3. The method of claim 1, wherein the extraction method provides water soluble extracts that have one or more well-defined and consistent spectra.

4. The method of claim 1, including using the FR-IR spectroscopy to identify and analyze minor components of the sample that contribute to the sample's flavor.

5. The method of claim 1, wherein the extraction method includes preparing the sample using water, chloroform, and ethanol.

6. A method for evaluating a sample cheese comprising determining one or more IR spectra of the cheese at least one point in time, comprising:
i) extracting one or more water-soluble components from the sample;
ii) precipitating complex proteins from the water soluble components to obtain a supernatant portion; and,
iii) conducting an FT-IR analysis on the supernatant portion.

7. The method of claim 6, including extracting in step i) with organic solvents to enable removal of compounds that interfere with detection of one or more compounds that contribute to the flavor.

8. The method of claim 7, wherein the detected compounds include one or more of organic acids and amino acids.

9. A method for evaluating a sample cheese comprising determining one or more IR spectra of the cheese at least one point in time, comprising:
i) mixing ground pieces of the sample with water; optionally sonicating the mixture to break down clumps of the sample;
ii) adding chloroform to the sample-water mixture of step i) sufficient to separate a supernatant comprising complex fat in the sample from remaining portions of the sample;
iii) mixing the resultant supernatant with ethanol sufficient to precipitate complex proteins from the supernatant to provide a test sample;
iv) centrifuging the resultant test sample; and
v) using the centrifuged test sample in a FT-IR analysis.

10. The method of claim 1, wherein FT-IR analysis detects one or more of:
asymmetric and symmetric stretching vibrations of C—H groups in methylene groups of long-chain fatty acids in the region 3,000 to 2,800 cm⁻¹;
signals from C═O groups of fatty acid esters at 1,740 cm⁻¹;
amide and amide II bands of proteins at around 1,640 and 1,540 cm⁻¹; and
bands in the region 1,800 to 900 cm⁻¹, due to C—H bending, C—O—H in-plane bending, and/or C—O stretching vibrations of lipids, organic acids, amino acids, and carbohydrate derivatives.

11. The method of claim 1, further including using the FR-IR spectra to correlate specific flavor notes such as fermented, sour, and unclean, and differentiated using multivariate classification models.

12. The method of claim 1, wherein the analysis time, including sample preparation time, is about 20 minutes or less, per sample.

13. The method of claim 1, wherein an expected final flavor quality of cheese early in the ripening process is determined at about 30 days of sample age or less.

14. The method of claim 1, further including detect differences in the composition of cheese from different production plants.

15. The method of claim 1, wherein the specific flavor notes are correlated to the infrared spectra and the cheese is classified based on its flavor quality.

16. The method of claim 1, wherein the method simultaneously determines one or more of: age, pH, fat, moisture, salt, and flavor quality of the cheese.

17. The method of claim 1, further including preparing the sample cheese by: i) grinding the sample with liquid nitrogen; ii) extracting and centrifuging the sample, and iii) collecting a water soluble fraction.

18. The method of claim 1, further including:
drying of an extract of the sample to result in formation of a uniform film of sample; and
collecting the FTIR spectra of the extract in a mid-infrared region, optionally using a 3-bounce zinc selenide ATR crystal.

19. The method of claim 1, wherein the FTIR spectra reflect the total chemical composition of the cheese extract, with absorbance bands due to acids, esters, alcohols and peptides.

20. The method of claim 1, wherein raw spectra data are transformed into second derivatives to remove baseline shifts, improve band resolution, and reduce noise and variability between replicates.

21. A method for providing quality control and control over the ripening process of cheese to achieve a cheese of desired flavor quality, comprising evaluating using the method of claim 1.

22. A method for classifying cheese based on its flavor quality, comprising using the method of claim 1.

23. A method for determining marketability of cheese early in a ripening process of the cheese, comprising using the method of claim 1.
